(19) United States
(12) Patent Application Publication
(10) Pub. No.: US 2016/0270647 A1
(43) Pub. Date: Sep. 22, 2016

(54) SYSTEM, METHOD AND COMPUTER-ACCESSIBLE MEDIUM FOR DETERMINING INFLAMMATION ASSOCIATED WITH A CENTRAL NERVOUS SYSTEM

(71) Applicant: THE GENERAL HOSPITAL CORPORATION, Boston, MA (US)

(72) Inventors: Charles P. LIN, Cambridge, MA (US); Clemens ALT, Watertown, MA (US)

(21) Appl. No.: 15/031,972

(22) PCT Filed: Oct. 27, 2014
(86) PCT No.: PCT/US14/62381

§ 371 Date: Apr. 25, 2016

Related U.S. Application Data
(60) Provisional application No. 61/895,749, filed on Oct. 25, 2013.

(51) Int. Cl. A61B 3/00; A61B 3/12; A61B 3/13; A61B 3/14; A61B 3/10; A61B 5/00

(52) U.S. Cl. CPC A61B 3/0025; A61B 3/102; A61B 5/4064; A61B 5/407; A61B 3/13; A61B 3/14; A61B 3/1241; A61B 3/1025

(57) ABSTRACT

An exemplary system, method and computer-accessible medium can be provided that can, for example, can be provided so as to receive regarding at least one portion of an ophthalmic sample(s) based on a radiation(s) provided from the sample(s). In addition, it is possible to determine whether an inflammation marker(s) is present in a portion(s) of the sample(s) based on the information. Further, an identification can be performed as to that an abnormality(s) exists in a further anatomical structure based on the determination. The further anatomical structure can be different from the sample(s).

### CROSS-REFERENCE TO RELATED APPLICATION(S)

This application relates to and claims priority from U.S. Patent Application No. 61/895,749, filed on Oct. 25, 2013, the entire disclosure of which is incorporated herein by reference.

### FIELD OF THE DISCLOSURE

The present disclosure relates generally to an assessment of central nervous system inflammation, and more specifically, to exemplary embodiments of systems, methods and computer-accessible media for assessing nervous system inflammation using, e.g., leukocyte-endothelial interaction in the retina.

### BACKGROUND INFORMATION

Inflammation is the immune reaction of a tissue to injury or infection that often involves an increase in blood flow with an influx of white blood cells (leukocytes) and chemokines that facilitate healing. Leukocyte infiltration can be a hallmark of inflammation, and a leukocyte-endothelial interaction (“LEI”) can be a first step in the recruitment of leukocytes from the circulation to the inflamed tissue. Therefore, a method to image LEI noninvasively can be useful for rapid assessment of the time course of brain injury and response to intervention. A method to detect LEI can be especially useful where sample collection and analysis (e.g., blood or spinal fluid) can be difficult, and access to heavy imaging machinery, such as MRI, may not be available, such as in a military setting or in rural, underserved areas.

Traumatic brain injury (“TBI”) can usually be accompanied, and can be exacerbated, by inflammation of or in a central nervous system (“CNS”). Survivors of TBI suffer from long-term disabilities, and even mild TBI can cause cognitive impairment, fatigue and pain. In experimental models of TBI, it has been shown that integrin on leukocytes and soluble intercellular cellular adhesion molecules (“ICAM”) can be elevated in TBI. Furthermore, aggregates of leukocytes and platelets can be found within hours after TBI, indicating that activation of leukocytes and platelets can be among the earliest signs of neuro-inflammation. Activated leukocytes can interact with endothelial cells that may themselves not express inflammatory signals, as demonstrated in leukocytes extravasating in the contralateral (e.g., uninjured) brain hemisphere after experimental TBI.

Similarly, multiple sclerosis is an autoimmune disease that is characterized by inflammation in the brain and spinal chord. For example, infiltration of the brain by autoreactive immune cells that originate in the peripheral circulation damages the axonal myelin sheath, thus resulting in a demyelination of the neurons. Demyelination interferes with a neuronal signal transmission, which in turn results in a number of physical or cognitive disabilities. Since myelin once damaged likely cannot be repaired, it is important to combat multiple sclerosis as early as possible.

Thus, it may be beneficial to provide an exemplary system, method and computer-accessible medium that can overcome at least some of the deficiencies described herein above, and provide, for example, an assessment of a nervous system inflammation.

### SUMMARY OF EXEMPLARY EMBODIMENTS

In reviewing a radiation-induced injury in the mouse retina, persistent inflammation (e.g., microglia activation, breakdown of blood-retinal barrier, and LEI) following a single dose of gamma irradiation was observed. Microglia activation was also visualized after controlled focal damage in retinal blood vessels using a CX3CR1 reporter mouse whose microglial cells (e.g., the resident immune sentinels of the CNS) express the green fluorescent protein (“GFP”). (See, e.g., FIG. 8A). LEI was observed by fluorescence or label-free backscattering contrast with a scanning laser ophthalmoscope (“SLO”), (see, e.g., FIGS. 8B and 8C), and was most prominent near the optic disc where the axons connect to the brain. LEI can occur only under inflammatory conditions, and can be absent in healthy retina.

For example, using exemplary embodiments of systems, methods and computer-accessible mediums according to the present disclosure, activated leukocytes, either autoreactive or after activation through interaction with the inflamed blood-brain barrier, can be detected interacting in the retinal vasculature by in vivo imaging. The integrity of the blood-retina barrier can be compromised by LEI, and become detectable by leakage of fluorescein. Thus, according to such exemplary embodiments, it is possible to characterize LEI and integrity of the blood-retina barrier in retinal vasculature in, e.g., autoimmune encephalomyelitis (EAE), a rodent model of MS, as potential markers that can be assessed by noninvasive imaging of the retina. It is also possible to determine if a decrease of clinical score during treatment, for example, with integrin alpha4 blocker (natalizumab) corresponds to a reduction of LEI and fluorescein leakage.

The inflammation associated with injury in the CNS, e.g., the eye, brain, spinal chord, etc., as well as in other locations and/or areas of anatomy can be assessed by imaging LEI, e.g., in the retina near the optic disc. For example, after an induction of inflammation in the brain, for example in TBI, Multiple Sclerosis (MS), a number of activated leukocytes remain in circulation for some time because not all leukocyte can infiltrate the site of inflammation at once. With an increasing circulation time, the probability that leukocytes pass the retinal vasculature, where they can be detected, can increase. Likewise, LEI associated with ocular diseases, such as diabetic retinopathy or Glaucoma, can be detected by an exemplary retinal imaging procedure according to an exemplary embodiment of the present disclosure. A retinal flow cytometer was previously developed for detection and quantification of fluorescently labeled leukocytes in the circulation of live animals. The exemplary systems, methods and computer-accessible mediums, according to an exemplary embodiment of the present disclosure, can be used to visualize activated leukocytes interacting with the retinal vasculature following CNS injury.

Exemplary embodiments of the systems, methods and computer accessible medium according to the present disclosure can be provided to quantify LEI in the retina and in the brain using an established model of radiation-induced CNS inflammation. Chimeric mice can be generated whose leukocytes can express the red fluorescent protein (“DsRed”), and whose microglia expresses the green fluorescent protein (“GFP”). LEI in the retinal vasculature can be assessed using an SLO developed specifically for mouse eye imaging, while LEI in the brain vasculature can be imaged through the thinned skull using a custom-built video rate laser scanning confocal/multiphoton microscope.

Microglia activation can serve as an independent marker for inflammation at these two locations. The exemplary systems, methods and computer-accessible mediums, according to an exemplary embodiment of the present disclosure, can be used to assess LEI following TBI. For example, the correlation (e.g., kinetics and dose response) between LEI in the retina and in the brain can be examined using the Marmarou model of TBI, or by controlled cortical impact.

An exemplary procedure using the exemplary SLO can be performed for human eye imaging. The SLO can be optimized for high resolution label free imaging of the optic disc by implementing adaptive optics and speckle reduction techniques. The optimum wavelength for imaging leukocyte based on intrinsic backscattering contrast can also be determined. Pilot studies to image LEI in human eyes can be initiated that can use optical coherence tomography (“OCT”) to image the retinal vasculature as a biomarker for TBI.

These and other objects of the present disclosure can be achieved by provisions of exemplary system, method and computer-accessible medium according to exemplary embodiments of the present disclosure that can, for example, receive regarding at least one portion of ophthalmic sample(s) based on a radiation(s) provided from the sample, determine whether an inflammation marker(s) is present in the portion(s) of the sample based on the received information, and identify that an abnormality(s) exists in a further anatomical structure based on the determination. The further anatomical structure can be different from the sample(s).

According to further exemplary embodiments of the present disclosure, the further structure can include a portion(s) of a central nervous system. For example, the radiation(s) can be provided from the retina of the sample(s). Imaging the retina of the sample(s) can be performed, and the determination can be made regarding the retina based on the image. The marker(s) can be measurable, and can include an interaction of white blood cells with a blood vessel wall. The marker(s) can also be or include an identification of blood vessel leakage. The information can be obtained from a confocal reflectance system, a fluorescence system, an optical coherence tomography system, or an optical frequency domain imaging system. In certain embodiments of the present disclosure the abnormality(s) can include (i) a brain injury, (ii) a spinal cord injury, (iii) multiple sclerosis, (iv) a stroke, or (v) a brain tumor. The abnormality(s) can also include (i) a brain abnormality, (ii) a spinal cord abnormality, (iii) or an ophthalmic abnormality.

These and other objects, features and advantages of the exemplary embodiments of the present disclosure will become apparent upon reading the following detailed description of the exemplary embodiments of the present disclosure, when taken in conjunction with the appended claims.

### BRIEF DESCRIPTION OF THE DRAWINGS

Further objects, features and advantages of the present disclosure will become apparent from the following detailed description taken in conjunction with the accompanying Figures showing illustrative embodiments of the present disclosure, in which:

FIG. 1 is a diagram of an exemplary scanning laser ophthalmoscope arrangement/system according to an exemplary embodiment of the present disclosure;

FIGS. 2A-2C are exemplary reflectance images obtained using an exemplary system, method and/or computer-accessible medium according to an exemplary embodiment of the present disclosure;

FIGS. 3A-3D are exemplary images of a retinae of a mouse obtained using the exemplary system, method and/or computer-accessible medium according to an exemplary embodiment of the present disclosure;

FIG. 3E is a graph of an exemplary quantification of cell populations according to an exemplary embodiment of the present disclosure;

FIG. 3F is a graph of an exemplary radius of the engrafting of bone marrow derived cells according to an exemplary embodiment of the present disclosure;

FIG. 4 is an exemplary image which was generated using a fluorescence retinal imaging procedure according to an exemplary embodiment of the present disclosure, and shows microglia engineered to express the green fluorescent protein, bone marrow derived cells expressing DsRed; vasculature labeled with Alexa647;

FIGS. 5A-5C are images of exemplary signs of inflammation, such as fluorescein leakage in compromised vasculature (5A), activation of microglia labeled with green fluorescent protein and LEI of bone leukocytes expressing DsRed (5B), LEI captured in reflectance mode without fluorescent markers;

FIGS. 6A and 6B are images of exemplary leukocyte-endothelial interactions obtained using the exemplary system, method and/or computer-accessible medium according to an exemplary embodiment of the present disclosure;

FIG. 6C is a graph of exemplary observations of leukocyte-endothelial interactions according to an exemplary embodiment of the present disclosure;

FIGS. 7A and 7B are images of an exemplary fluorescein angiography obtained using the exemplary system, method and/or computer-accessible medium according to an exemplary embodiment of the present disclosure;

FIG. 7C is a graph illustrating the evaluation of exemplary image contrasts according to an exemplary embodiment of the present disclosure;

FIGS. 8A-8C are images of exemplary microglia activation and LEI according to an exemplary embodiment of the present disclosure;

FIG. 9 is a block diagram of an exemplary system in accordance with certain exemplary embodiments of the present disclosure; and

FIG. 10 is a flow diagram of a method in according to particular exemplary embodiments of the present disclosure which can be performed by one or more exemplary systems/arrangements/apparatus described and/or shown herein.

Throughout the drawings, the same reference numerals and characters, unless otherwise stated, are used to denote like features, elements, components, or portions of the illustrated embodiments. Moreover, while the present disclosure will now be described in detail with reference to the figures, it is done so in connection with the illustrative embodiments and is not limited by the particular embodiments illustrated in the figures or in the appended claims.

### DETAILED DESCRIPTION OF EXEMPLARY EMBODIMENTS

The exemplary system, method and computer-accessible medium, according to an exemplary embodiment of the present disclosure, can facilitate a longitudinal in-vivo tracking of both the resident microglial (e.g., disappearing) and the BMDC (e.g., engrafting) populations that can assist to obtain a more complete picture of cellular radiation response. For example, the inner retina, as an optically-directly accessible gray matter compartment of the CNS, lends itself to in vivo imaging and cell tracking, facilitating disease progression in individual animals to be followed serially over time. The exemplary system, method and computer-accessible medium according to an exemplary embodiment of the present disclosure can utilize a multi-color SLO specifically for confocal imaging of the mouse retina with the compatibility of simultaneously acquiring up to three fluorescence channels at video rate. The dynamics of the native microglia population and the engrafting BMDCs after BMT have been investigated. For this purpose, heterozygous CX3CR1 mice that express GFP in retinal microglia were exposed to a lethal dose of gamma radiation and rescued with a bone marrow transplant from universal DsRed donor mice. The numbers of native GFP+ cells and donor DsRed+ BMDCs in the retina were quantified by in vivo imaging before and over a time course of four months after the irradiation and BMT. Progressive loss of GFP+ microglia was observed with delayed engraftment of DsRed+ BMDCs. The total cell number was below the baseline value of resident microglial cells for most of the observation period, even after four months. Leukocyte endothelial interaction, which can be essential for circulating cells to recognize their homing site, was observed throughout this period, suggesting prolonged inflammation in the retina. Fluorescein angiography demonstrated that the integrity of the blood-retina barrier can be compromised after irradiation.

Exemplary Results

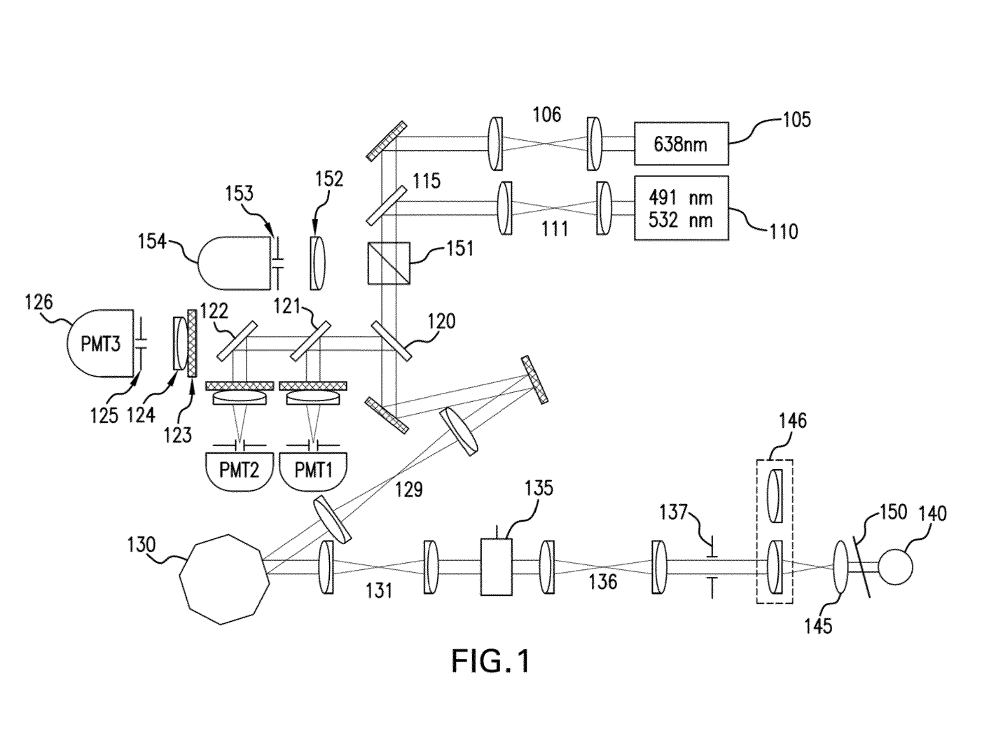

An exemplary SLO has been developed for a retinal imaging of a mammal (e.g., mouse, human, etc.) based on the video-rate confocal microscope. (See, e.g., References 15 and 16). Multiple laser sources and a multi-edge dichroic beam splitter facilitated up to three channels to be acquired simultaneously (See, e.g., FIG. 1). For example, as shown in FIG. 1, the exemplary SLO according to exemplary embodiments of the present disclosure can have, e.g., three or more laser sources at about 638 nm (e.g., L4 635S-24/OSYS, Micro Laser Systems, Inc.) 105 and at about 491 and about 532 nm (e.g., Dual Calypso, Cobolt AB) 110. The laser beams can be alternately focused and recollimated by one or more (and, e.g., numerous) lenses and the beam path folded by mirrors. The laser beams can be combined by, e.g., an approximately 580 nm long-pass dichroic beam splitter (e.g., Semrock) 115, and directed through a central, multi-edge dichroic beam splitter (e.g., Di01-T488/5321638, Semrock) 120. A telescope 129 can compress the diameter of the laser beam. An exemplary multiple (e.g., 36) facet polygon scanner 130 can be used to scan the beam horizontally at, e.g., about 17,280 lines per second, and a galvanometer 135 can provide the vertical scan. These pupils of the scan engine can be conjugated and relayed by telescopes 131, 136 into a system pupil 137, that can be imaged into the iris of an eye 140 (e.g., an eye of a mouse, human, etc.). In the exemplary SLO, the cornea and lens of the eye serve as the objective and the image quality critically can depend on the optical characteristics of the eye. The exemplary tube lens can be or include a 60D Volk lens 145. If a mouse is being evaluated, it can be held in a tube mounted on a six-axis stage that can facilitate precise positioning of the mouse eye pupil into the system pupil formed by the Volk lens 145. The mouse and the Volk lens can be moved together relative to the last lens 146 to adjust the focus within the retina. The following lens can be interchangeable to facilitate changing the field of view between, e.g., about 15° and about 45° (e.g., approximately 400 to 1,200 μm), covering the central to mid-periphery regions of the mouse retina.

Reflectance imaging can be accomplished using, e.g., a polarization rotation with a quarter-wave plate 150 placed between the Volk lens 145 and the eye 140. Further, a polarizing beam splitter cube 151 can separate backscattering from the retina from the incident light and direct it onto a focusing lens 152 that focuses the reflected light through a confocal pinhole 153 into the reflectance detector 154. The fluorescence emitted in the retina of the eye 140 can be separated from the excitation light, and directed into the fluorescence detection arm by the main, triple-edge dichroic beam splitter. Inside the fluorescence detection arm, e.g., 560 nm and 650 longpass dichroic beam splitters at 560 nm (e.g., FF560-Di01, Semrock) 121 and 650 nm (e.g., FF650-Di01, Semrock) 122 can separate light into three (or more) distinct fluorescence detectors. Each fluorescence detector can be or include an assembly including, e.g., a bandpass filter 123 to further narrow the fluorescence detection (e.g., red=650-825 nm, green=550-650 nm and blue=500-550 nm) and an approximately 75 mm achromatic lens 124 that can focus the light through an approximately 50 μm confocal pinhole 125 (e.g., corresponding to about 3.2 to 4.1 times the Airy disc size) into a photomultiplier tube (“PMTs”) 126 (e.g., R3896). Three or more channels, for example, can be acquired simultaneously when the PMTs 126 and 154 are connected to one or more computers, thereby, e.g., facilitating the observation of up to three distinct cell populations in real-time at video-rate. It should be understood that other type of electro-magnetic radiations (e.g., other than light) can be used with the exemplary embodiments described herein. In addition, the above-described configuration is merely exemplary, and it should be understood that other configuration can be implemented in accordance with the exemplary embodiments of the present disclosure.

Pinholes of about 50 μm in diameter, corresponding to 3 to 4 Airy discs, resulted in a depth of focus of approximately 40 μm, thus facilitating a thick optical section of retinal tissue to be imaged at once, without the need for axial movement. In its current configuration, the SLO does not use adaptive optics. (See, e.g., References 17, 18). GFP and DsRed were excited with the 491 and 532 nm excitation laser(s) (e.g., see exemplary configuration 110 of FIG. 1). The fluorescence thereof was detected through 525/50 and 593/40 (e.g., both Semrock) bandpass filters and assigned green and red color in the AGB images, respectively. Alexa647 for staining vasculature was excited by the 638 nm laser (e.g., see exemplary configuration 105 of FIG. 1), detected through a 650 nm longpass filter and assigned the blue channel.

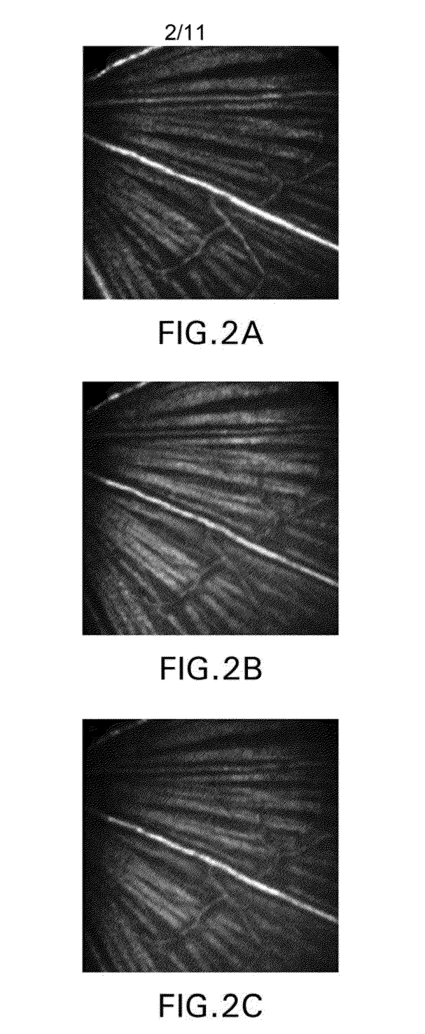

An alignment of the exemplary (e.g., laser or other electro-magnetic radiation producing) sources 105, 110 and exemplary respective telescopes 106, 111 can be undertaken to reduce or minimize chromatic focusing error(s) among the multiple (e.g., three) wavelengths. To verify the results of the exemplary system alignment, the inner retina was imaged in reflectance mode with each of the three lasers in the same mouse. The exemplary results indicate that the three lasers image the same optical section in the mouse retina (See e.g., exemplary reflectance images shown in FIG. 2).

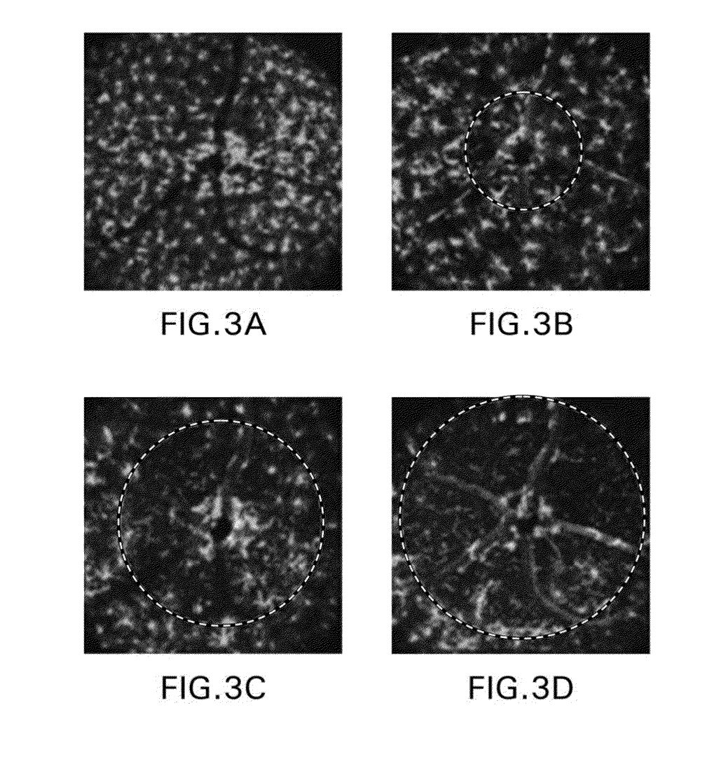

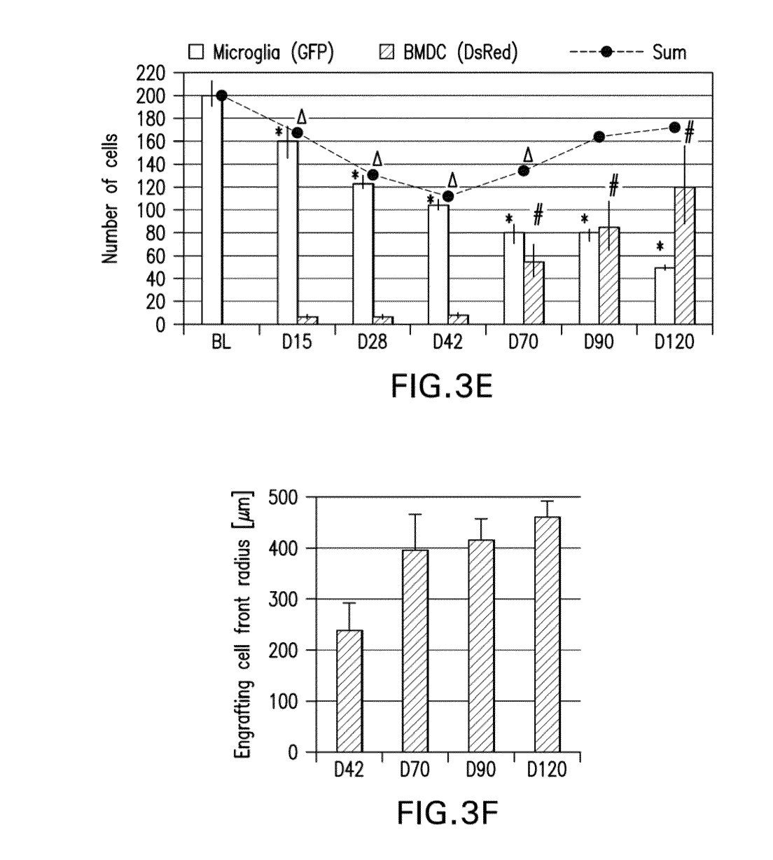

GFP+ resident microglia and DsRed+ BMDCs were tracked over a time course of four months after BMT from universal DsRed donors into lethally irradiated CX3CR1 recipient mice (n=3). FIGS. 3A-3D show representative exemplary images of one mouse followed over the 120-day experimental period. Each exemplary image of FIGS. 3A-3D shows a field of view of 30° (e.g., approximately 800 μm) centered on the optic nerve head, where FIG. 3A shows a baseline image taken prior to any procedure, FIG. 3B illustrates an image taken 42 days after the irradiation and bone marrow transplant, and FIG. 3C is an exemplary image taken 70 and 3D was taken 90 days after irradiation and bone marrow transplant. Qualitatively, progressive loss of endogenous GFP+ microglia (shown as light patterns in FIGS. 3A-3D, e.g., not shown as lines) and influx of transplanted DsRed+ cells (shown as darker bright patterns in FIGS. 3B-3D, some, e.g., some shown as lines extending from the center) were observed over time. Most DsRed+ cells circulated in the vasculature because all newly generated hematopoietic cells were derived from the DsRed+ donor, but closer examination indicates that some of the DsRed+ cells were stationary outside the vasculature (e.g., in the retina parenchyma). Quantitatively, the baseline number of native microglia was about 202±12 cells.

This number decreased by 0% at the first measured time point 15 days after irradiation and transplantation (See e.g., exemplary graph of FIG. 3E). By 42 days, the number of microglia had declined to 50% of the baseline number measured prior to irradiation. Over the same time course, negligible numbers (e.g., on average less than about 10 cells per field of view) of extravasating BMDCs were detected, resulting in a relatively depleted hematopoietic cell population in the retina. Substantial engraftment (e.g., 56±15 cells per field of view) was first observed at day 70. At the end of the observation period, at day 120, the number of native GFP+ microglia decreased to 25% of the baseline level, while the DsRed+ cells continued to increase to 122±34 cells per field of view. Due to delayed BMDC engraftment, the total number of hematopoietic cells, that is, the sum of all remaining GFP+ microglia and DsRed+ BMDCs, was relatively depleted and approached 85% of the baseline number of microglia after four months. Engrafting BMDCs were initially observed near the optic nerve head; the radius of the BMDC repopulation front increased over time (See e.g., exemplary graph of FIG. 3F).

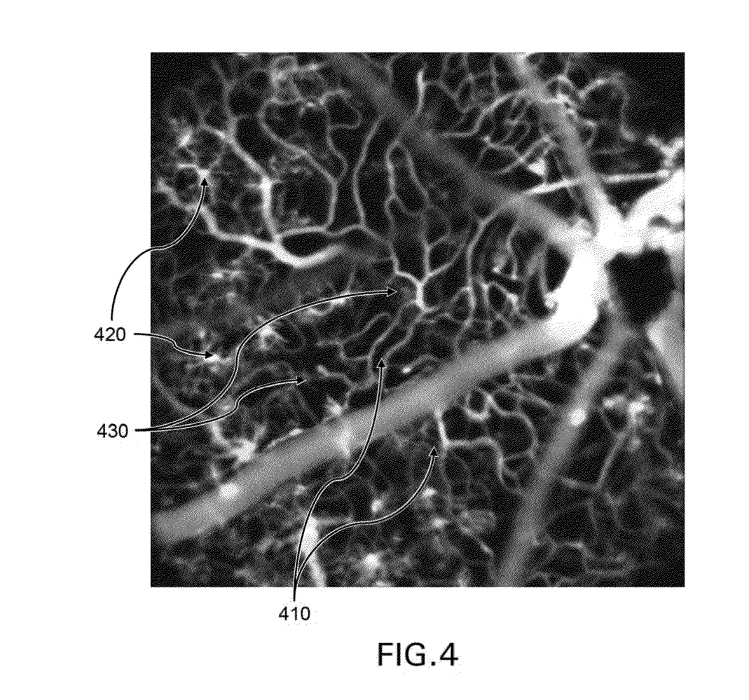

FIG. 4 illustrates an exemplary image which was generated using a fluorescence retinal imaging procedure according to an exemplary embodiment of the present disclosure. Such exemplary image can be produced with, e.g., the exemplary scanning laser ophthalmoscope (SLO) according to an exemplary embodiment of the present disclosure, which can effectuate, e.g., retinal imaging in any subject, including a mouse or any other mammal. SLO according the exemplary embodiment of the present disclosure can resolve retinal microvasculature (410) and individual cells in the retina, such as, e.g., microglia (420) (e.g., lighter cells) and bone marrow derived cells (430) (e.g., darker and brighter cells).

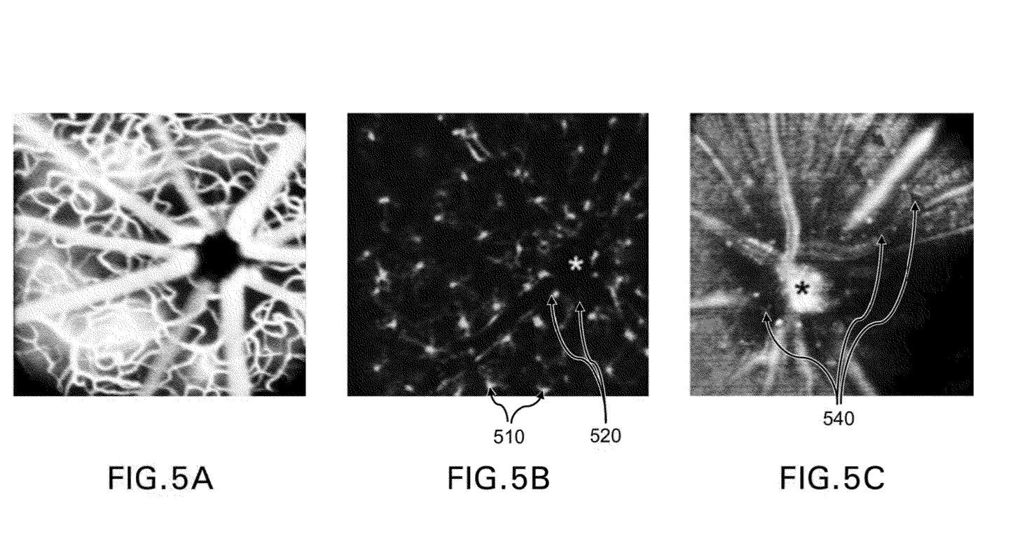

A breakdown of the blood-retina barrier can be and was detected by an exemplary fluorescein angiography procedure, where fluorescein leaks into the retinal parenchyma were present, e.g., only where the BRB is damaged (as shown in FIG. 5A—which shows an image of a Leakage through compromised blood retinal barrier 30 seconds after the injection of the fluorescein becomes apparent as a loss of contrast between retinal microvasculature and parenchyma). LEI was observed by fluorescence or label-free backscattering contrast with a scanning laser ophthalmoscope (SLO) (as shown in FIGS. 5B and 5C). Indeed, FIG. 5B illustrates resident microglia labeled with GFP (lighterspots 510) and rolling leukocytes labeled with DsRed (darker spots 520) seven (7) days after gamma radiation. FIG. 5C shows an exemplary image of rolling leukocytes (lighterspots pointed to by arrows 540) which can be observed by backscattering contrast (e.g., reflectance with no fluorescent label), whereas an asterisk marks the optic nerve head. For example, LEI occurs only under inflammatory conditions, and is absent in healthy retina and is most prominent near the optic disc.

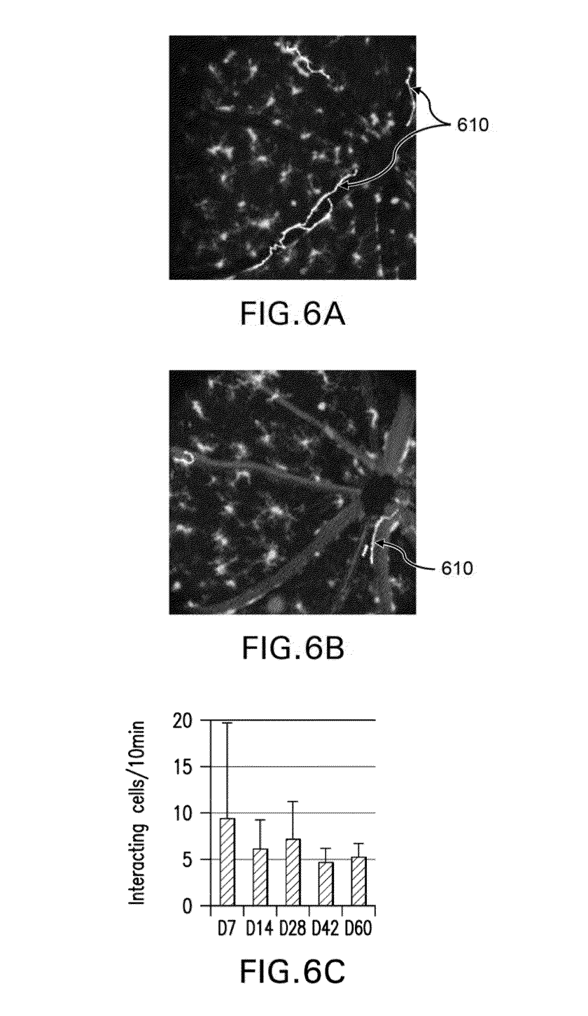

One of the important aspects of in vivo imaging can be the ability to detect dynamic interactions between sells and their environment. Thus, in addition to longitudinal cell tracking, in vivo imaging can facilitate “zooming in” on the short-term dynamics at each time point to visualize cell behavior and cellular interactions that may not be available using histological methods. Circulating BMDCs that can temporarily interact with the vascular endothelium can be observed by acquiring time-lapse images. FIGS. 6A-6C illustrate exemplary results of an exemplary manually tracking DsRed+ leukocytes after time-lapse imaging, where one 10-frame average image was acquired every 30 seconds over a period of about ten minutes. The tracking marks the path individual moving leukocytes have taken (610)—e.g., white tracks shown in FIGS. 6A and 6B. The tracks marked by the tracking indicate that the leukocytes move unidirectionally along the vasculature after one week after irradiation (as shown in FIG. 6A). Subsequently, the tracks sometimes fold, indicating that the moving cells change direction within the same blood vessel, meaning the cells may move against the blood flow (as shown in FIG. 6B). Repeated time-lapse imaging can demonstrate that leukocyte endothelial interaction, normally absent in healthy retinal vasculatures, can occur throughout the observation period in time-lapse imaging, even two months after the irradiation. Enumeration of the interacting cells (as shown in FIG. 6C) can demonstrate that after an initial peak at day 7 the number of interacting cells can remain at approximately 5 cells per 10 min observation period for the duration of the experiment. The interacting leukocytes can move with an average velocity on the order of about 10 μm/min. The duration of interaction can last up to about 600 sec.

Exemplary Discussion

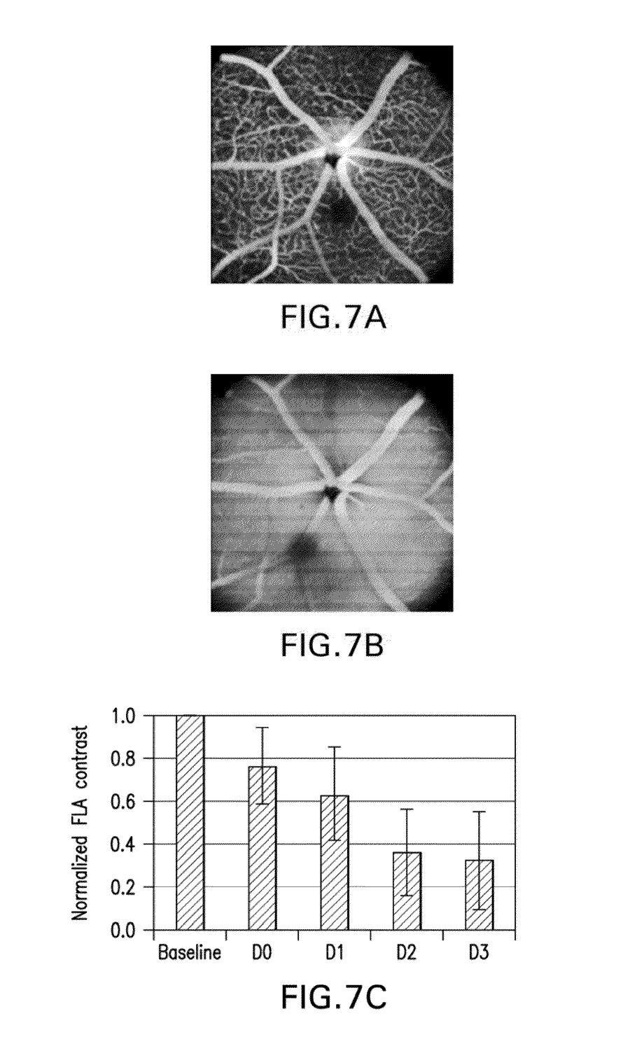

Consistent with the notion of inflammation, e.g., leukocyte endothelial interaction can be observed after the irradiation and bone marrow transplant. Leukocyte endothelial interaction can be the result of adhesion molecule mediated signaling that can enable circulating leukocytes to roll, arrest and eventually extravasate near the site of an inflammation. (See, e.g., Reference 31). Adhesion molecule upregulation and leukocyte endothelial interaction can be considered to last approximately one week after injury. (See, e.g., References 32 and 33). While a peak of interacting leukocytes was observed seven days after irradiation, the exemplary results can indicate that leukocyte endothelial interaction continues throughout the observation period. The interaction of leukocytes and endothelial cells can be frequently accompanied by a disruption of the blood-retinal barrier. Fluorescein angiography can demonstrate that the blood retinal barrier (BRB) was compromised during the first few days after irradiation. (See, e.g., FIGS. 7A-7C). For example, prior to irradiation, the intact BRB can confine fluorescein to the vasculature, and can prevent leakage out of the blood vessels, so that a sharp contrast between vasculature and parenchyma is observed (as illustrated in FIG. 7A). When the BRB is compromised, fluorescein can leak out of the blood vessels into the retinal parenchyma, minimizing image contrast between vasculature and parenchyma (as shown in FIG. 7B). The change in image contrast can serve as an indicator to quantify how severely the BRB has been disrupted. FIG. 7C shows that contrast decreases during the first three days after irradiation, indicating that leakage over this timecourse increases.

In the protected environment of the CNS and the retina, e.g., leukocyte endothelial interaction and leakage through the blood-retina barrier may not be observed under physiological condition, but can be considered to be signs of inflammation. (See, e.g., References 34 and 35). For example, a subset of resident monocytes can also patrol the intact vasculature of the mesentery and brain under physiological conditions without extravasating. (See, e.g., References 36 and 37). Thus, a fraction of LEI at later time points can be a fact physiological LEI of such resident monocytes.

In vivo imaging can provide long-term and short-term dynamic information regarding, e.g., the behavior and interactions of cells that cannot be gathered with ex vivo methods. The exemplary system, method and computer-accessible medium according to an exemplary embodiment of the present disclosure can track and/or quantify the endogenous microglia and engrafting BMDC populations simultaneously over months in the living mouse retina by in vivo retinal imaging. For example, an engraftment of DsRed+ BMDC after lethal irradiation and bone marrow transplant in CX3CR1GFP/+ mice can be accompanied by loss of the resident GFP+ microglia. Leukocyte endothelial interaction, thought to be absent under homeostatic conditions and commonly associated with CNS inflammation, can be observed even months after the irradiation. It is possible to directly correlate the effects of ionizing radiation on retinal vascular integrity, microglia and BMDCs in dependence of the irradiation dose directly delivered to the head.

Exemplary Materials and Methods

The exemplary system according to the present disclosure has been described with reference to FIG. 1

Attention to the axial alignment of the imaging lasers can be provided to compensate for the chromatic aberrations of, e.g., the mouse eye that have been reported to be approximately 7D across the visible wavelength range. (See, e.g., Reference 25). The instrument can initially be aligned with the red laser as a reference beam. The exemplary lengths of the various telescopes can be optimized to minimize divergence and times-diffraction-limit factor of the reference beam as measured with a beam propagation analyzer (e.g., ModeMaster, Coherent). The confocal pinhole of the reflectance channel can be conjugated by placing a mirror in the last intermediate image plane of the system and optimizing the confocal throughput. To match the focal plane of the other laser wavelengths (e.g., about 488 and about 532 nm) to that of the red laser, the respective source telescope can be slightly adjusted to introduce a small beam divergence. The exemplary alignment of the three laser beams can be optimized in three dimensions in an artificial eye, built from a 2 mm focal length lens (e.g., NA=0.5, 2 mm clear aperture, Geltech 350150) held in a brass housing with a target placed in the focal plane of the lens. An exemplary source telescope of the 491 and 532 laser can be adjusted until best possible images of the target can be acquired with all three wavelengths. As an additional alignment procedure, the confocal pinholes of the three fluorescence channels can be conjugated for each excitation wavelength.

Mice expressing GFP in microglia under the control of the fractalkine receptor promoter CX3CR1 (e.g., B6.129P-Cx3cr1tm1Litt/J) were purchased from Jackson Laboratory. The fractalkine receptor can be specifically expressed on microglia, a population of blood monocytes, NK and dendritic cells. (See, e.g., Reference 38, 18). The mice were maintained as heterozygotes by crossing homozygous CX3CR1-GFP mice with the parental C57BL/6 strain to ensure proper function of the fractalkine receptor on microglia. (See, e.g., Reference 39). Mice were exposed to a single dose of 9 Gy gamma radiation with a Cesium source (e.g., Gammacell 40 Exactor, MDS Nordion). Lethally irradiated mice were rescued five hours after the exposure by bone marrow transplantation of 4×106 cells harvested from homozygous actin-DsRed donor mice [B6. Cg-Tg(CAG-DsRed*MST)1Nagy/J].

For the exemplary imaging procedure, the mice were held in a heated holding tube that integrated a nose cone for delivery of 1-2% isoflurane mixed in oxygen for inhalation anesthesia. The tube was mounted on a six-axis stage that aided the positioning of the mouse eye in the SLO imaging beam. The pupil was dilated with a drop of Tropicamide. A contact lens was placed on the mydriatic eye and a drop of GenTeal eye drops prevented the cornea from drying. In vivo images were recorded at baseline prior to the irradiation and at days 15, 28, 42, 70, 90 and 120 after the irradiation. At each time point, the numbers of resident GFP+ cells and DsRed+ bone marrow derived cells were evaluated.

Exemplary Image Analysis

To identify interactions between bone marrow derived leukocytes with the blood vessel endothelial wall, exemplary time-lapse imaging procedure(s) can be performed. One exemplary image can be taken, e.g., once every 30 sec for several minutes. The exemplary images of the resulting temporal stack can be aligned to compensate for motion artifacts such as rotation or drifting of the eye. Cells moving in the major blood vessels can be tracked, e.g., manually in ImageJ using the MTrackJ plugin, or automatically.

Exemplary Fluorescein Angiography

C57BL/6 mice were injected with 25 μl of 5% fluorescein solution, diluted in Phosphate Buffered Saline from 10% Fluorescein (e.g., USP, IMS Ltd.) via the tail vein while under isofluorane anesthesia and on the SLO stage. Immediately, images were taken at 30 sec intervals over a 3 min period to follow leakage of the fluorescein into the retinal parenchyma. As a measure of fluorescein leakage, the contrast between capillaries and non-vascular tissue was evaluated within segments delineated by the major blood vessels. Michelson contrast, defined as the ratio of the difference between maximum and minimum pixel values over their sum, was used for the measurement. Contrast value was normalized to baseline values taken before irradiation for each mouse.

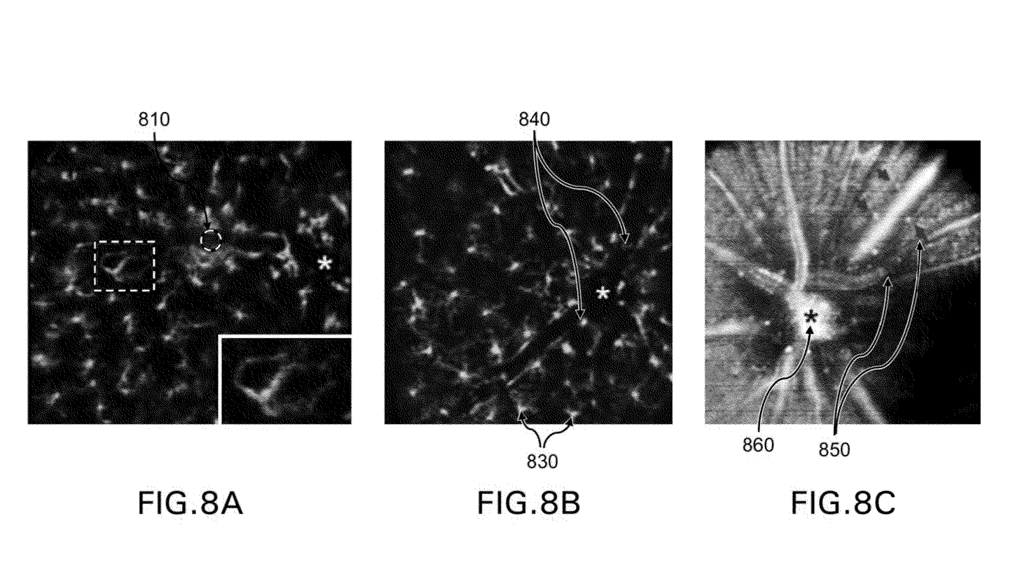

FIG. 8A illustrates an exemplary image obtained using the exemplary system, method and/or computer-accessible medium according to another exemplary embodiment of the present disclosure. For example, such exemplary image emphasizes a focal laser injury in retinal vasculature (see dashed circle 810) that causes an activation of nearby microglia as characterized by their polarization towards the injury site and accumulation of cells around the injured vessel. The inset shown in FIG. 8A illustrates an exemplary magnification of the cell enclosed by the dashed rectangle. FIG. 8B shows an exemplary image obtained using such exemplary system, method and/or computer-accessible medium with resident GFP plus microglia (lighter dots 830) and rolling DsRed plus leukocytes (darker dots 840), e.g., 7 days after gamma radiation. Further, FIG. 8C illustrates an exemplary image of rolling leukocytes (lighter arrows 850) can be observed by backscattering contrast (no fluorescent label). It is noted that the asterisk 860 marks the optic nerve head, which was obtained using the exemplary system, method and/or computer-accessible medium according to the present disclosure.

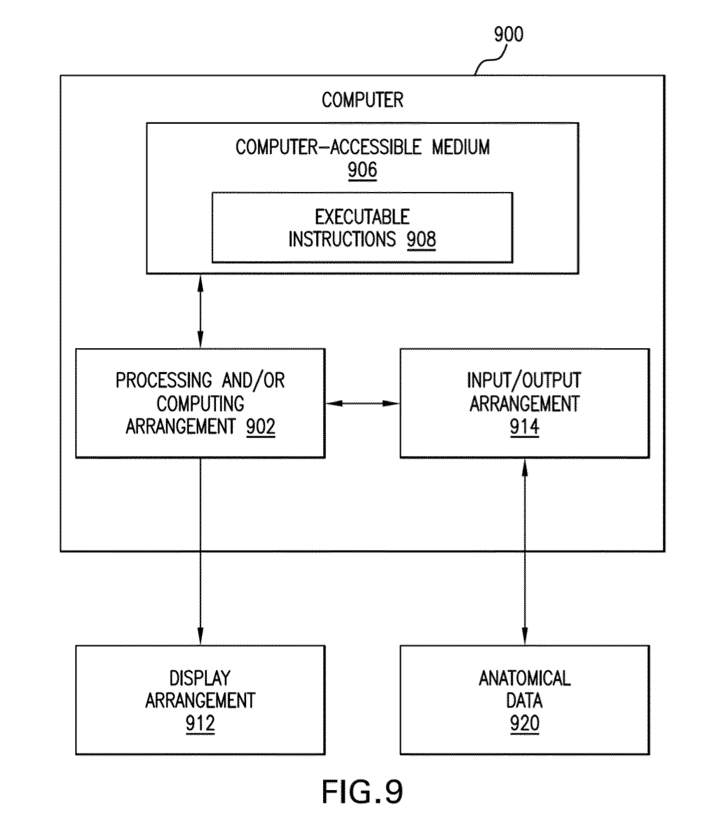

FIG. 9 shows a block diagram of an exemplary embodiment of a system according to the present disclosure, which can implement the exemplary embodiments of the method and procedures described herein. For example, exemplary procedures in accordance with the present disclosure described herein can be performed by a processing arrangement and/or a computing arrangement 902. Such processing/computing arrangement 902 can be, for example, entirely or a part of, or include, but not limited to, a computer/processor 904 that can include, for example, one or more microprocessors, and use instructions stored on a computer-accessible medium (e.g., RAM, ROM, hard drive, or other storage device).

As shown in FIG. 9, for example, a computer-accessible medium 906 (e.g., as described herein above, a storage device such as a hard disk, floppy disk, memory stick, CD-ROM, RAM, ROM, etc., or a collection thereof) can be provided (e.g., in communication with the processing arrangement 902). The computer-accessible medium 906 can contain executable instructions 908 thereon. In addition or alternatively, a storage arrangement 910 can be provided separately from the computer-accessible medium 906, which can provide the instructions to the processing arrangement 902 so as to configure the processing arrangement to execute certain exemplary procedures, processes and methods, as described herein above, for example.

Further, the exemplary processing arrangement 902 can be provided with or include an input/output arrangement 914, which can include, for example, a wired network, a wireless network, the internet, an intranet, a data collection probe, a sensor, etc. For example, anatomical data 920 can be provided to the input/output arrangement 914. As shown in FIG. 9, the exemplary processing arrangement 902 can be in communication with an exemplary display arrangement 912, which, according to certain exemplary embodiments of the present disclosure, can be a touch-screen configured for inputting information to the processing arrangement in addition to outputting information from the processing arrangement, for example. Further, the exemplary display 912 and/or a storage arrangement 910 can be used to display and/or store data in a user-accessible format and/or user-readable format.

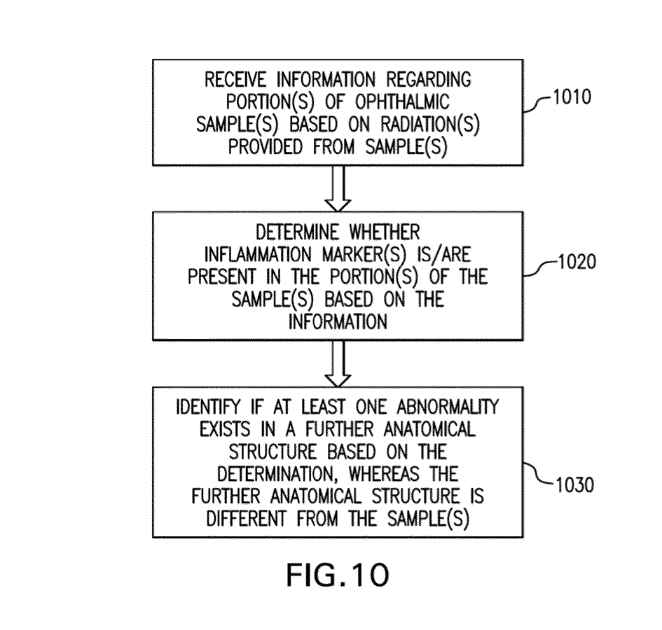

FIG. 10 illustrates a flow diagram of the method according to another exemplary embodiment of the present disclosure, which can be executed by any of the exemplary systems, computer-accessible medium and apparatus described herein. For example, information regarding at least one portion of ophthalmic sample(s) can be received based on a radiation(s) provided from the sample(s) (procedure 1010). Then, it can be determined whether inflammation marker(s) is/are present in such portion(s) of the sample(s) based on the information (procedure 1020). Further, in procedure 1030, it can be identified if one or more abnormalities exists in a further anatomical structure based on the determination of procedure 1020. The further anatomical structure can be different from the sample(s).

For example, the further structure can include a portion(s) of a central nervous system. The radiation(s) can be provided from the retina of the sample(s). Imaging the retina of the sample(s) can be performed, and the determination can be made regarding the retina based on the image. The marker(s) can be measurable, and can include an interaction of white blood cells with a blood vessel wall. The marker(s) can also include or be an identification of blood vessel leakage. The information regarding the portions of procedure 1010 can be obtained from a confocal reflectance system, a fluorescence system, an optical coherence tomography system, and/or an optical frequency domain imaging system. In certain embodiments of the present disclosure, the abnormality(s) can include (i) a brain injury, (ii) a spinal cord injury, (iii) multiple sclerosis, (iv) a stroke, or (v) a brain tumor. The abnormality(s) can also include (i) a brain abnormality, (ii) a spinal cord abnormality, (iii) or an ophthalmic abnormality.

The foregoing merely illustrates the principles of the disclosure. Various modifications and alterations to the described embodiments will be apparent to those skilled in the art in view of the teachings herein. It will thus be appreciated that those skilled in the art will be able to devise numerous systems, arrangements, and procedures which, although not explicitly shown or described herein, embody the principles of the disclosure and can be thus within the spirit and scope of the disclosure. Various different exemplary embodiments can be used together with one another, as well as interchangeably therewith, as should be understood by those having ordinary skill in the art. In addition, certain terms used in the present disclosure, including the specification, drawings and claims thereof, can be used synonymously in certain instances, including, but not limited to, for example, data and information. It should be understood that, while these words, and/or other words that can be synonymous to one another, can be used synonymously herein, that there can be instances when such words can be intended to not be used synonymously. Further, to the extent that the prior art knowledge has not been explicitly incorporated by reference herein above, it is explicitly incorporated herein in its entirety. All publications referenced are incorporated herein by reference in their entireties.

### EXEMPLARY REFERENCES

The following references are hereby incorporated by reference in their entirety.
1. Hari P, Pasquini M C, Vesole D H. Cure of multiple myeloma—more hype, less reality. Bone Marrow Transplant 2006; 37:1-18; PMID:16258534.
2. Moon S J, Mieler W F. Retinal complications of bone marrow and solid organ transplantation. Curr Opin Ophthalmol 2003; 14:433-42; PMID:14615651; http://dx.doi.org/10.1097/00055735-200312000-00018.
3. Chung H, Kim K H, Kim J G, Lee S Y, Yoon Y H. Retinal complications in patients with solid organ or bone marrow transplantations. Transplantation 2007; 83:694-9; PMID:17414700; http://dx.doi.org/10.1097/01.tp.0000259386.59375.8a.
4. Coskuncan N M, Jabs D A, Dunn J P, Haller J A, Green W R, Vogelsang G B, et al. The eye in bone marrow transplantation. VI. Retinal complications. Arch Ophthalmol 1994; 112:372-9; PMID:8129664; http://dx.doi.org/10.1001/archopht.1994.01090150102031.
5. Giuliari G P, Sadaka A, Hinkle D M, Simpson E R. Current treatments for radiation retinopathy. Acta Oncol 2011; 50:6-13; PMID:20722590; http://dx.doi.org/10.3109/0284186X.2010.500299.
6. Chen M, Zhao J, Luo C, Pandi S P, Penalva R G, Fitzgerald D C, et al. Para-inflammation-mediated retinal recruitment of bone marrow-derived myeloid cells following whole-body irradiation is CCL2 dependent. Glia 2012; 60:833-42; PMID:22362506; http://dx.doi.org/10.1002/glia.22315.
7. Mildner A, Schmidt H, Nitsche M, Merkler D, Hanisch U K, Mack M, et al. Microglia in the adult brain arise from Ly-6ChiCCR2+ monocytes only under defined host conditions. Nat Neurosci 2007; 10:1544-53; PMID: 18026096; http://dx.doi.org/10.1038/nn2015.
8. Simard A R, Rivest S. Bone marrow stem cells have the ability to populate the entire central nervous system into fully differentiated parenchymal microglia. FASEB J 2004; 18:998-1000; PMID:15084516.
9. Ono K, Takii T, Onozaki K, Ikawa M, Okabe M, Sawada M. Migration of exogenous immature hematopoietic cells into adult mouse brain parenchyma under GFP-expressing bone marrow chimera. Biochem Biophys Res Commun 1999; 262:610-4; PMID: 10471372; http://dx.doLorg/10.1006/bbrc.1999.1223.
10. Xu H, Chen M, Mayer E J, Forrester J V, Dick A D. Turnover of resident retinal microglia in the normal adult mouse. Glia 2007; 55:1189-98; PMID:17600341; http://dx.doi.org/10.1002/glia.20535.
11. Vilhardt F. Microglia: phagocyte and glia cell. Int J Biochem Cell Biol 2005; 37:17-21; PMID:15381143; http://dx.doi.org/10.1016/j.bioce1.2004.06.010.
12. Nimmerjahn A, Kirchhoff F, Helmchen F. Resting microglial cells are highly dynamic surveillants of brain parenchyma in vivo. Science 2005; 308:1314-8; PMID:15831717; http://dx.doi.org/10.1126/science.1110647.
13. Davalos D, Grutzendler J, Yang G, Kim J V, Zuo Y, Jung S, et al. ATP mediates rapid microglial response to local brain injury in vivo. Nat Neurosci 2005; 8:752-8; PMID:15895084; http://dx.doi.org/10.1038/nn1472.
14. Ajami B, Bennett J L, Krieger C, Tetzlaff W, Rossi F M V. Local self-renewal can sustain CNS microglia maintenance and function throughout adult life. Nat Neurosci 2007; 10:1538-43; PMID:18026097; http://dx.doi.org/10.1038/nn2014.
15. Veilleux I, Spencer J S, Biss D P, Cote D, Lin C P. In Vivo Cell Tracking With Video Rate Multimodality Laser Scanning Microscopy. IEEE J Sel Top Quant Elec 2008; 14: 10-18.
16. Rajadhyaksha M, Gonzalez S, Zavislan J M, Anderson R R, Webb R H. In vivo confocal scanning laser microscopy of human skin II: advances in instrumentation and comparison with histology. J Invest Dermatol 1999; 113:293-303; PMID:10469324; http://dx.doi.org/10.1046/j.1523-1747.1999.00690.x.
17. Biss D P, Sumorok D, Burns S A, Webb R H, Zhou Y, Bifano T G, et al. In vivo fluorescent imaging of the mouse retina using adaptive optics. Opt Lett 2007; 32:659-61; PMID:17308593; http://dx.doi.org/10.1364/OL.32.000659.
18. Alt C, Biss D P, Tajouri N, Jakobs T C, Lin C P. An adaptive-optics scanning laser ophthalmoscope for imaging murine retinal microstructure. Proc SPIE 2010; 7550:755019; http://dx.doi.org/10.1117/12.840583.
19. Cordeiro M F, Guo L, Luong V, Harding G, Wang W, Jones H E, et al. Real-time imaging of single nerve cell apoptosis in retinal neurodegeneration. Proc Natl Acad Sci USA 2004; 101:13352-6; PMID:15340151; http://dx.doi.org/10.1073/pnas.0405479101.
20. Eter N, Engel D R, Meyer L, Helb H M, Roth F, Maurer J, et al. In vivo visualization of dendritic cells, macrophages, and microglial cells responding to laser-induced damage in the fundus of the eye. Invest Ophthalmol Vis Sci 2008; 49:3649-58; PMID:18316698; http://dx.doi.org/10.1167/iovs.07-1322.
21. Paques M, Simonutti M, Augustin S, Goupille O, El Mathari B, Sahel J A. In vivo observation of the locomotion of microglial cells in the retina. Glia 2010; 58:1663-8; PMID:20578032; http://dx.doi.org/10.1002/glia.21037.
22. Leung C K, Weinreb R N, Li Z W, Liu S, Lindsey J D, Choi N, et al. Long-term in vivo imaging and measurement of dendritic shrinkage of retinal ganglion cells. Invest Ophthalmol Vis Sci 2011; 52:1539-47; PMID:21245394; http://dx.doi.org/10.1167/iovs.10-6012.
23. Alt C, Lin C P. In vivo quantification of microglia dynamics with a scanning laser ophthalmoscope in a mouse model of focal laser injury. Proc SPIE 2012; 8209:820907; http://dx.doi.org/10.1117/12.909141.
24. de la Cera E G, Rodriguez G, Llorente L, Schaeffel F, Marcos S. Optical aberrations in the mouse eye. Vision Res 2006; 46:2546-53; PMID:16516259; http://dx.doi.org/10.1016/j.visres.2006.01.011.
25. Geng Y, Schery L A, Sharma R, Dubra A, Ahmad K, Libby R T, et al. Optical properties of the mouse eye. Biomed Opt Express 2011; 2:717-38; PMID:21483598; http://dx.doi.org/10.1364/BOE.2.000717.
26. Geng Y, Greenberg K P, Wolfe R, Gray D C, Hunter J J, Dubra A, et al. In vivo imaging of microscopic structures in the rat retina. Invest Ophthalmol Vis Sci 2009; 50:5872-9; PMID: 19578019; http://dx.doi.org/10.1167/iovs.09-3675.
27. Geng Y, Dubra A, Yin L, Merigan W H, Sharma R, Libby R T, et al, Adaptive optics retinal imaging in the living mouse eye. Biomed Opt Express 2012; 3:715-34; PMID:22574260; http://dx.doi.org/10.1364/BOE.3.000715.
28. Nakano N, Ikeda H O, Hangai M, Muraoka Y, Toda Y, Kakizuka A, et al. Longitudinal and simultaneous imaging of retinal ganglion cells and inner retinal layers in a mouse model of glaucoma induced by N-methyl-D-aspartate. Invest Ophthalmol Vis Sci 2011; 52:8754-62; PMID:22003119; http://dx.doi.org/10.1167/iovs.10-6654.
29. Burns S A, Zhangyi Z, Chui T Y P, Song H, Elsner A E, Malinovsky V E. Imaging the Inner Retina Using Adaptive Optics. Invest Ophthalmol Vis Sci 2008; 49:4512.
30. Tam J, Martin J A, Roorda A. Noninvasive visualization and analysis of parafoveal capillaries in humans. Invest Ophthalmol Vis Sci 2010; 51:1691-8; PMID:19907024; http://dx.doi.org/10.1167/iovs.09-4483.
31. Crane I J, Liversidge J. Mechanisms of leukocyte migration across the blood-retina barrier. Semin Immunopathol 2008; 30:165-77; PMID:18305941; http://dx.doi.org/10.1007/s00281-008-0106-7.
32, Yuan H, Goetz D J, Gaber M W, Issekutz A C, Merchant T E, Kiani M F. Radiation-induced up-regulation of adhesion molecules in brain microvasculature and their modulation by dexamethasone. Radiat Res 2005; 163:544-51; PMID:15850416; http://dx.doi.org/10.1667/RR3361.
33. Gaber M W, Yuan H, Killmar J T, Naimark M D, Kiani M F, Merchant T E. An intravital microscopy study of radiation-induced changes in permeability and leukocyte-endothelial cell interactions in the microvessels of the rat pia mater and cremaster muscle. Brain Res Brain Res Protoc 2004; 13:1-10; PMID:15063835; http://dx.doi.org/10.1016/j.brainresprot.2003.11.005.
34. Jain P, Coisne C, Enzmann G, Rottapel R, Engelhardt B. Alpha4beta1 integrin mediates the recruitment of immature dendritic cells across the blood-brain barrier during experimental autoimmune encephalomyelitis. J Immunol 2010; 184:7196-206; PMID:20483748; http://dx.doi.org/10.4049/jimmunol.0901404.
35. Pachter J S, de Vries H E, Fabry Z. The blood-brain barrier and its role in immune privilege in the central nervous system. J Neuropathol Exp Neurol 2003; 62:593-604; PMID:12834104.
36. Auffray C, Fogg D, Garfa M, Elain G, Join-Lambert O, Kayal S, et al. Monitoring of blood vessels and tissues by a population of monocytes with patrolling behavior. Science 2007; 317:666-70; PMID:17673663; http://dx.doi.org/10.1126/science.1142883.
37. Audoy-Remus J, Richard J F, Soulet D, Zhou H, Kubes P, Vallières L. Rod-Shaped monocytes patrol the brain vasculature and give rise to perivascular macrophages under the influence of proinflammatory cytokines and angiopoietin-2. J Neurosci 2008; 28:10187-99; PMID:18842879; http://dx.doi.org/10.1523/JNEUROSCI.3510-08.2008.
38. Jung S, Aliberti J, Graemmel P, Sunshine M J, Kreutzberg G W, Sher A, et al. Analysis of fractalkine receptor CX(3)CR1 function by targeted deletion and green fluorescent protein reporter gene insertion. Mol Cell Biol 2000; 20:4106-14; PMID:10805752; http://dx.doi.org/10.1128/MCB.20.11.4106-4114.2000.
39. Liang K J, Lee J E, Wang Y D, Ma W, Fontainhas A M, Fariss R N, et al. Regulation of dynamic behavior of retinal microglia by CX3CR1 signaling. Invest Ophthalmol Vis Sci 2009; 50:4444-51; PMID:19443728; http://dx.doi.org/10.1167/iovs.08-3357.
40. Alt, C., Runnels, J. M., TEO, G. S. L. & Lin, C. P. In vivo tracking of hematopoietic cells in the retina of chimeric mice with a scanning laser ophthalmoscope. intravital 1, (2012).

1. A method, comprising:
receiving information regarding at least one portion of at least one ophthalmic sample based on at least one radiation provided from the sample;
determining whether at least one inflammation marker is present in the at least one portion of the at least one sample based on the information; and
with a computer arrangement, identifying that least one abnormality exists in a further anatomical structure based on the determination, wherein the further anatomical structure is different from the at least one sample.

2. The method according to claim 1, wherein the further structure includes at least one portion of a central nervous system.

3. The method according to claim 1, wherein the at least one radiation is provided from a retina of the at least one sample.

4. The method according to claim 4, further comprising imaging the retina of the at least one sample, wherein the determination is made regarding the retina based on the image.

5. The method according to claim 1, wherein the at least one marker is measurable.

6. The method according to claim 1, wherein the at least one marker includes an interaction of white blood cells with a blood vessel wall.

7. The method according to claim 1, wherein the at least one marker includes an identification of a blood vessel leakage.

8. The method according to claim 1, wherein the information is obtained from a confocal reflectance system.

9. The method according to claim 1, wherein the information is obtained from a fluorescence system.

10. The method according to claim 1, wherein the information is obtained from an optical coherence tomography system.

11. The method according to claim 1, wherein the information is obtained from an optical frequency domain imaging system.

12. The method according to claim 1, wherein the at least one abnormality includes at least one of (i) a brain injury, (ii) a spinal cord injury, (iii) multiple sclerosis, (iv) a stroke, or (v) a brain tumor.

13. The method according to claim 1, wherein the at least one abnormality includes at least one of (i) a brain abnormality, (ii) a spinal cord abnormality or, (iii) an ophthalmic abnormality.

14. The method according to claim 1, wherein the at least one inflammation marker includes a leukocyte.

15. The method according to claim 1, wherein the imaging procedure includes imaging a leukocyte-endothelial interaction within the retina.

16. A system comprising:
a processor that is configured and programmed to:
receive information regarding at least one portion of at least one ophthalmic sample based on at least one radiation provided from the at least one sample;
determine whether at least one inflammation marker is present in the at least one portion of the at least one sample based on the information; and
identify that at least one abnormality exists in a further anatomical structure based on the determination, wherein the further anatomical structure is different from the at least one sample.

17. The system according to claim 16, wherein the further structure includes at least one portion of a central nervous system.

18. The system according to claim 16, wherein the at least one radiation is provided from a retina of the at least one sample.

19. The system according to claim 18, wherein the processor is further configured and programmed to image the retina of the at least one sample, and wherein the determination is made regarding the retina by the processor based on the image.

20. The system according to claim 16, wherein the at least one marker is at least one of (i) measurable, or (ii) includes an identification of a blood vessel leakage, or of a blood leakage.

21.-22. (canceled)

23. The system according to claim 16, wherein the information is obtained from at least one of a confocal reflectance system, a fluorescence system, an optical coherence tomography system, or an optical frequency domain imaging system.

24-26. (canceled)

27. The system according to claim 16, wherein the at least one abnormality includes at least one of (i) a brain injury, (ii) a spinal cord injury, (iii) multiple sclerosis, (iv) a stroke, (v) a brain tumor, (vi) a brain abnormality, (vii) a spinal cord abnormality, or (viii) an ophthalmic abnormality.

28. (canceled)

29. The system according to claim 16, wherein the at least one inflammation marker includes a leukocyte.

30. The system according to claim 16, wherein the processor images the retina by imaging a leukocyte-endothelial interaction within the retina.

31. A non-transitory computer-accessible medium which includes software instructions, wherein, when a processer executes the instructions, the processor is configured to execute procedures comprising:
receiving information regarding at least one portion of at least one ophthalmic sample based on at least one radiation provided from the at least one sample;
determining whether at least one inflammation marker is present in the at least one portion of the at least one sample based on the information; and
identifying that at least one abnormality exists in a further anatomical structure based on the determination, wherein the further anatomical structure is different from the at least one sample.
